(19) United States
(12) Patent Application Publication
(10) Pub. No.: US 2005/0042196 A1
(43) Pub. Date: Feb. 24, 2005

(54) Antimicrobial phenol containing cyanoacrylate compositions

(76) Inventors: Ian Askill, Colorado Springs, CO (US); Michael J. Rezsofi, Denver, CO (US); Herbert H. Gary, Monroe Township, NJ (US); A. Sidney Finkelstein, Stamford, CT (US)

Correspondence Address: FOLEY & LARDNER LLP;THREE PALO ALTO SQUARE, 3000 EL CAMINO REAL, SUITE 100, PALO ALTO, CA, 94306, US

(21) Appl. No.: 10/859,927

(22) Filed: Jun. 2, 2004

Related U.S. Application Data
(60) Provisional application No. 60/475,857, filed on Jun. 3, 2003.
(60) Provisional application No. 60/490,309, filed on Jul. 25, 2003.
(60) Provisional application No. 60/498,913, filed on Aug. 29, 2003.

(51) Int. Cl. A61K 31/785; C08K 5/05

(52) U.S. Cl. 424/78.27; 524/384

(57) ABSTRACT

Disclosed are methods and formulations for the treatment or prevention of infections on mammalian tissues such as skin. Specifically, the methods of this invention involve the in situ formation of a polymeric cyanoacrylate film containing phenol over mammalian tissue.

### CROSS-REFERENCE TO RELATED CASES

This application claims the benefit under 35 U.S.C. §119(e) of the following U.S. Provisional Patent Applications: U.S. Provisional Patent Application Ser. No. 60/475,857 filed Jun. 3, 2003; U.S. Provisional Patent Application Ser. No. 60/490,309 filed Jul. 25, 2003; and U.S. Provisional Patent Application Ser. No. 60/498,913 filed Aug. 29, 2003 all of which applications are incorporated herein by reference in their entirety.

### BACKGROUND OF THE INVENTION

### Field of the Invention

This invention is directed to methods and formulations for the treatment or prevention of infections on mammalian tissues such as skin. Specifically, the methods of this invention involve the in situ formation of a polymeric cyanoacrylate film over mammalian tissue wherein the film comprises an antimicrobially effective amount of phenol.

### References

The following patent applications, and patents are cited in this application as superscript numbers:

1 Barley, Methods for Retarding Blister Formation by Use of Cyanoacrylate Adhesives, U.S. Pat. No. 5,306,490, issued Apr. 26, 1994;

2 Barley, et al., Methods for Treating Suturable Wounds by Use of Sutures and Cyanoacrylate Adhesives, U.S. Pat. No. 5,254,132 issued Oct. 19, 1993;

3 McIntire, et al., Process for the Preparation of Poly(α-Cyanoacrylates), U.S. Pat. No. 3,654,239, issued Apr. 4, 1972;

4 Barley, et al., Methods for Treating Non-Suturable Wounds by Use of Cyanoacrylate Adhesives, U.S. Pat. No. 6,342,213, issued Jan. 29, 2002;

5 Barley, et al., Methods for Reducing Skin Irritation from Artificial Devices by Use of Cyanoacrylate Adhesives, U.S. Pat. No. 5,653,789, issued Aug. 5, 1997;

6 Tighe, et al., Methods for Inhibiting Skin Ulceration by Use of Cyanoacrylate Adhesives, U.S. Pat. No. 5,403,591 issued Apr. 4, 1995;

7 Tighe, et al., Use of Cyanoacrylates for Providing a Protective Barrier, U.S. Pat. No. 5,580,565, issued Dec. 6, 1996;

8 Askill, et al., Methods for Draping Surgical Incision Sites, U.S. Pat. No. 5,807,563 issued Sep. 15, 1998;

9 Greff, et al., Cyanoacrylate Compositions Comprising an Antimicrobial Agent, U.S. Pat. No. 5,684,042, issued Nov. 3, 1997;

10 McDonnell, et al., Sterilized Cyanoacrylate Adhesive Composition, and a Method of Making Such a Composition, U.S. Pat. No. 5,530,037, issued Jun. 25, 1996; and

11 Nicholson, et al., Cyanoacrylate Solutions Containing Preservatives, PCT Publication WO 01/32795, published May 10, 2001.

All of the above patent applications and patents and all other publications, patents, and patent applications referenced in this application are incorporated herein by reference in their entirety to the same extent as if each individual publication, patent application or patent was specifically and individually indicated to be incorporated by reference in its entirety.

### State of the Art

Many commercial topical antibiotic preparations exist in creams, lotions, petroleum bases, etc. While they are easily applied, they are also easily rubbed off (e.g., ointments applied to the skin frequently rub off onto the patient's clothing within minutes or hours thereby losing their effectiveness). This problem is most commonly addressed by applying protective layers or covers such as dressings over the ointments.

The covers, while simple to use, can absorb the ointment or cause it to displace to another area. The ointment can also prevent the adhesive on the dressing from effectively adhering. In some cases, attempts have been made to incorporate the active ingredients in pre-formed films or in film-forming solutions. Unfortunately, most preformed films have limited diffusion of the active ingredients and show little or no clinical activity.

Greff, et at.⁹ have demonstrated that certain iodophors can be incorporated into prepolymeric cyanoacrylate compositions to create stable film forming liquids wherein the iodophor effectively provides for antimicrobial activity to the polymer film formed therefrom. However, many antimicrobial agents are incompatible with prepolymeric cyanoacrylate compositions causing either immediate polymerization or preventing polymerization from occurring at all or within a reasonable period of time after application to mammalian tissue.

Certain of these iodophors are also insoluble in the cyanoacrylate composition thereby necessitating thorough mixing of the composition immediately prior to application. That is to say that during the normal shelf-life of the composition, the insoluble iodophor suspended in the composition will fall out of suspension thereby necessitating mixing of the composition immediately prior to use.

In addition, the incorporation of an iodophor to the composition transforms the composition from clear or transparent to a reddish solution. When applied to skin, the reddish formed polymer layer can be unsightly.

Prepolymeric cyanoacrylate compositions have been disclosed for use in a variety of medical environments such as an alternative or adjunct to sutures² or as a hemostat³. Other described uses of cyanoacrylate prepolymers include their use on mammalian tissue to form polymeric films that are utilized:

1 to prevent friction blister formation¹,

2 in treating small non-suturable wounds⁴,

3 in inhibiting surface skin irritation arising from friction between the skin surface and artificial devices such as tapes, prosthetic devices, casts, etc.⁵,

4 as surgical incise drapes⁸,

5 in inhibiting skin ulceration⁶, and

6 forming a protective film to inhibit skin degradation due to incontinence⁷.

In each case, the combination of an antimicrobial agent with these compositions would be useful particularly as a replacement for conventional bandages. Furthermore, the use of a soluble, color-free, antimicrobial agent would provide for a more elegant and user compatible product. Still further, compositions having enhanced shelf-life would provide for easy to use products over prolonged periods of time.

### SUMMARY OF THE INVENTION

It has now been discovered that the antimicrobial agent phenol may be successfully incorporated into a topical cyanoacrylate adhesive composition providing a synergistic increase in stability without inducing any adverse side affects to the tissue to which it has been applied. While phenol has long been known to act as a polymerization inhibitor for cyanoacrylate adhesives¹⁰, the specific use of phenol as an antimicrobial agent in topical medical applications and the resulting synergistic stabilizing effect in cyanoacrylate prepolymeric compositions was not previously known. The suitability of phenol as an antimicrobial agent in topical adhesives is also surprising as phenol is a known caustic agent and it was unknown if extended contact with a tissue would result in tissue irritation and/or incompatibility with other conventional additives employed in the composition.

The invention described in this patent is directed, in part, to cyanoacrylate adhesive compositions comprising polymerizable cyanoacrylate ester, phenol, polymerization inhibitors, and an optional biocompatible plasticizer. These compositions are useful in methods for covering mammalian tissue and, in particular, mammalian skin with a polymeric antimicrobial film that may reduce the risk of infection to the underlying and/or adjacent tissue and, in the case of mammalian skin, may form a waterproof film over the skin.

The invention described herein is also directed, in part, to methods for the treatment or prevention of infections in mammalian tissue which methods involve formation of an antimicrobial cyanoacrylate polymeric film over mammalian tissue by the in situ polymerization of the cyanoacrylate adhesive composition. This composition can be applied as a liquid/gel to the tissue surface and can include additional therapeutic agents such as analgesics, anti-inflammatory agents, and the like.

Additional advantages and novel features of the invention will be set forth, in part, in the description which follows, and, in part, will become apparent to those skilled in the art upon examination of the following, or may be learned by practice of the invention.

The cyanoacrylate adhesive compositions described herein generally include a polymerizable cyanoacrylate component; a phenol component, in an amount sufficient to provide effective antimicrobial activity; polymerization inhibitors, in amounts sufficient provide inhibition or retardation of polymerization prior to the use of the composition; and an optional biocompatible plasticizer component, in an amount sufficient to provide enhanced flexibility for the resulting film coating formed by polymerization of the cyanoacrylate adhesive composition.

In one version of the cyanoacrylate adhesive composition, the composition comprises:

(a) a polymerizable cyanoacrylate ester;

(b) about 0.01 to about 5 weight percent phenol based on the total weight of the composition;

(c) about 100 to about 3,500 ppm of polymerization inhibitors; and

(d) optionally from about 10 to about 30 weight percent based on the total weight of the composition of a biocompatible plasticizer.

Described in detail in the Detailed Description Section below are other ranges of the amount of phenol that may be used in the cyanoacrylate adhesive compositions described herein.

Preferably, the composition employs a blend of different cyanoacrylate esters in order to provide greater flexibility and wear durability, such as taught in Berger, et al., U.S. Pat. No. 5,998,472. A particularly preferred mixture comprises about 1.5:1 to about 2:1 mixture of n-butyl cyanoacrylate and octyl cyanoacrylate (e.g., 2-ethylhexyl cyanoacrylate). Another preferred mixture comprises about 1.5:1 to about 2:1 mixture of n-butyl cyanoacrylate and decyl cyanoacrylate.

The composition also preferably employs a blend of polymerization inhibitors, with blends of hydroquinone (HQ) and sulfur dioxide being preferred, and blends of hydroquinone, 4-methoxyphenol, and sulfur dioxide being most preferred. In a particularly preferred embodiment, these inhibitors are employed in a 4:4:1.5 ratio.

The cyanoacrylate adhesive compositions described herein may be used for the treatment or prevention of infections on mammalian tissues, particularly mammalian skin. When used in this manner, the method comprises:

(a) applying to bacterially infected mammalian tissue or mammalian tissue at risk of bacterial infection an amount of a cyanoacrylate adhesive composition sufficient to form an adherent polymeric film on the tissue where the composition was applied upon polymerization of the cyanoacrylate adhesive composition.

The polymerizable cyanoacrylate ester component of the composition generally contains one or more cyanoacrylate ester monomers.

Preferably, the polymerizable cyanoacrylate ester component contains one or more cyanoacrylate esters that, in monomeric form, are represented by formula I: 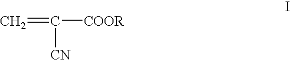
wherein:

R is selected from the group consisting of:
alkyl of 1 to 10 carbon atoms,
alkenyl of 2 to 10 carbon atoms,
cycloalkyl groups of from 5 to 8 carbon atoms,
phenyl,
2-ethoxyethyl,
3-methoxybutyl,
and a substituent of the formula: 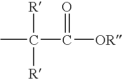
   wherein

each R′ is independently selected from the group consisting of:
hydrogen and methyl, and

R″ is selected from the group consisting of:
alkyl of from 1 to 6 carbon atoms,
alkenyl of from 2 to 6 carbon atoms,
alkynyl of from 2 to 6 carbon atoms,
cycloalkyl of from 3 to 8 carbon atoms,
aralkyl selected from the group consisting of benzyl,
methylbenzyl and phenylethyl,
phenyl, and
phenyl substituted with 1 to 3 substituents selected from the group
consisting of hydroxyl, chloro, bromo, nitro, alkyl of 1 to 4 carbon atoms,
and alkoxy of from 1 to 4 carbon atoms.

More preferably, in the cyanoacrylate esters of formula I, R is alkyl of from 2 to 10 carbon atoms and even more preferably alkyl of from 2 to 8 carbon atoms. Still more preferably, R is butyl, pentyl or octyl.

In a preferred version of the cyanoacrylate adhesive composition, the cyanoacrylate ester component contains a blend of two different cyanoacrylate monomers. One preferred blend that may be used contains a mixture of n-butyl cyanoacrylate and octyl cyanoacrylate, with 2-ethylhexyl cyanoacrylate being the preferred octyl cyanoacrylate.

In another preferred embodiment, the polymerized cyanoacrylate composition has a thickness of no more than about 1 millimeter and, more preferably, the polymer layer has a thickness of from about 2 to about 500 microns and still more preferably about 20 to about 1 00 microns.

### DETAILED DESCRIPTION OF THE PREFERRED EMBODIMENTS

Definitions

This invention is directed to methods and formulations for the treatment or prevention of infections on mammalian tissues such as skin.

Before this invention is described in detail, it is to be understood that, unless otherwise indicated, this invention is not limited to any particular composition, reactable cyanoacrylate ester, or biocompatible plasticizer, as such may vary. It is also to be understood that unless terms are specifically defined the terminology used herein is for the purpose of describing particular embodiments only and is not intended to limit the scope of the present invention.

In this specification and in the claims that follow, reference will be made to a number of terms that, unless the context makes obvious otherwise, shall be defined to have the following meanings:

The term “alkyl”, as used herein, refers to monovalent alkyl groups of 1 to 10 carbon atoms, which may be straight chained or branched, and include, for example, methyl, ethyl, propyl (—CH₂CH₂CH₃ or —CH(CH₃)CH₃), n-butyl, and the like.

The term “alkenyl”, as used herein, refers to monovalent straight or branched chain alkyl groups having from 2 to 10 carbon atoms and more preferably 2 to 6 carbon atoms and having at least 1 and preferably from 1-2 carbon-carbon double bonds. Examples include but are not limited to vinyl, prop-1-en-1-yl, allyl, —CH═C(CH₂CH₃)₂ and the like.

The term “alkynyl”, as used herein, refers to monovalent straight or branched chain alkyl groups having from 2 to 10 carbon atoms and more preferably 2 to 6 carbon atoms and having at least 1 and preferably from 1-2 carbon-carbon triple bonds. Examples include but are not limited to ethynyl, propynyl, —C═CCH(CH₃)₂ and the like.

The term “cycloalkyl”, as used herein, refers to a cyclized alkyl ring containing from 3 to 10 ring atoms, preferably 5 to 8 ring atoms and more preferably, 5 to 6 ring atoms. The cycloalkyl group may optionally be substituted with from 1 to 3 alkyl groups. Cycloalkyl is also intended to cover alkyl substituted cycloalkyl groups where the point of attachment to the core molecule is through the alkyl group on the cycloalkyl ring.

The term “alkoxy”, as used herein, refers to O-alkyl groups where alkyl is defined as herein.

The term “aralkyl”, as used herein, refers to -alkyl-aryl groups where alkyl and aryl are as defined herein. And this group is exemplified for example by benzyl, phenethyl, and the like.

The term “Aryl or “Ar”, as used herein, refers to an unsaturated aromatic carbocyclic group of from 6 to 14 carbon atoms having a single ring (e.g., phenyl) or multiple condensed rings (e.g., naphthyl or anthryl) which condensed rings may or may not be aromatic (e.g., benzo[1,3]dioxole-6-yl, benzo[b]thiophen-5-yl, chroman-4-one-6-yl, and the like) provided that the point of attachment is on an aromatic carbocyclic group. Preferred aryls include phenyl and naphthyl.

The term “biocompatible” refers to a material that, in the amounts used, is substantially non-toxic and substantially non-irritating when applied to tissue.

“cP” as used herein refers to a centipoise and is equivalent to a centistoke (cSt).

The term “antimicrobially effective amount” refers to an amount of an antimicrobial agent sufficient to ameliorate the symptoms of, prevent the symptoms of, or delay the progression of a microbial infection or other ailment.

The Cyanoacrylate Adhesive Composition

The cyanoacrylate adhesive compositions described herein generally contain a polymerizable cyanoacrylate ester component, phenol, a polymerization inhibitor or mixture of inhitors, and an optional biocompatible plasticizer. Below is a detailed description of the amounts and types of the different components that may be used.

Specifically, the adhesive compositions of the invention exhibit one or more of the following: enhanced antimicrobial properties, increased stability (as indicated by increased shelf-life), increased flexibility, increased adherence to mammalian skin an/or superior wear durability.

Inclusion of phenol, particularly in the amounts described below, in combination with the other components used in the compositions, unexpectedly provides for a synergistic increase in one or more of the above-mentioned properties of the adhesive composition while providing the desired antimicrobial properties.

The Polymerizable Cyanoacrylate Ester Component

The polymerizable cyanoacrylate ester components employed in the adhesive composition contain one or more cyanoacrylate monomers or polymerizable cyanoacrylate oligomers. In their monomeric form the cyanoacrylate monomers are preferably compounds represented by formula I as described above. The polymerizable cyanoacrylate ester component preferably comprise from about 50 to about 99.9 weight percent of the total weight of the adhesive composition, more preferably about 50 to about 99.5 weight percent of the total weight of the adhesive composition and even more preferably from about 75 to about 99.5 and still more preferably from about 75 to about 89.5 weight percent of the total weight of the adhesive composition.

In formula I, R is preferably an alkyl group from 2 to 10 carbon atoms including but not limited to ethyl, n-propyl, iso-propyl, n-butyl, iso-butyl, sec-butyl, n-pentyl, iso-pentyl, n-hexyl, iso-hexyl, 2-ethylhexyl, n-heptyl, octyl, nonyl, and decyl. More preferably, R is butyl, pentyl or octyl and most preferably, R is n-butyl or octyl, with octyl most preferably being 2-ethyl hexyl. Mixtures of such compounds can also be employed as disclosed by Berger, et al., U.S. Pat. No. 5,998,472, which is incorporated herein by reference in its entirety.

In a preferred embodiment, the polymerizable cyanoacrylate composition contains a blend of n-butyl cyanoacrylate and octyl cyanoacrylate. The ratio of n-butyl cyanoacrylate ester to octyl cyanoacrylate ester in this preferred blend preferably ranges from about 1.5:1 to about 2:1 (w/w). It has been found that this blend of cyanoacrylate esters provides improved flexibility to the resulting polymerized film and increases the adhesion and wear durability of the polymeric film.

In an alternative preferred embodiment, a blend of n-butyl cyanoacrylate and decyl cyanoacrylate may be used. The preferred ratio of n-butyl cyanoacrylate ester to decyl cyanoacrylate ester ranges from about 1.5:1 to about 2:1 (w/w). It has been found that this blend of cyanoacrylate esters also provides improved flexibility, increased adhesion, and enhanced wear durability to the resulting polymeric film.

In yet another preferred embodiment, a blend of n-butyl cyanoacrylate and ethoxyethyl cyanoacrylate may be used. The preferred ratio of n-butyl cyanoacrylate ester to ethoxyethyl cyanoacrylate ester ranges from about 1:1 to about 2:1 (w/w).

The polymerizable cyanoacrylate ester components described herein rapidly polymerize in the presence of water vapor or tissue protein and bond to mammalian skin tissue without causing histotoxicity or cytotoxicity.

Generally, the polymerizable cyanoacrylate ester component may contain one or more known cyanoacrylate ester monomers. Polymerizable cyanoacrylate ester monomers known in the art and are described in, for example, U.S. Pat. Nos. 3,527,224; 3,591,676; 3,667,472; 3,995,641; 4,035,334; and 4,650,826 the disclosures of each are incorporated herein by reference in their entirety.

Phenol

Generally, the of phenol in the cyanoacrylate adhesive composition is an antimicrobially effective amount and is a non-toxic amount. Using the teachings in this patent and techniques well known in the art, one of skill in the technology described herein would find it straightforward to determine the range of antimicrobially effective amounts of phenol and the range of non-toxic amounts of phenol. For example, measurements of minimum inhibition concentrations (MIC) or the measurement of zones of inhibition (ZOI) conferred by the films of this invention would be useful. All phenol amounts described here are as weight percentages of phenol based on the total weight of the composition. Ranges of amounts of phenol that may be used include from about 0.01% to about from about 0.01% to about 5%, from about 0.1% to about 1.4%, from about 0.1% to about 1.0%, from about 0.5% to about 1.0%, from about 0.6% to about 1.0% and from about 0.1% to about 0.6%. Phenol concentrations may also be as low as about 0.005%. The inventors have experimentally demonstrated or are led to believe by their experimental results combined with their knowledge of the technology that these ranges of phenol provide the above-discussed synergistic antimicrobial/stabilizin effect. While the use of phenol as an antimicrobial agent has been suggested by others, the synergistic stabilizing effect was not present in the adhesives so suggested.

Although phenol is a known caustic agent and is not recommended for extended contact with bare skin, it has surprisingly been found that cyanoacrylate adhesive formulations of this invention are substantially non-irritating and are suitable for use in these formulations.

The Polymerization Inhibitor

The cyanoacrylate adhesive compositions described herein also contain about 100 to about 3,500 ppm of polymerization inhibitor(s). The polymerization inhibitor(s) is (are) present in amounts effective to inhibit premature polymerization of the composition during storage. As used herein, unless the context makes obvious otherwise, the term “inhibiting amount” of a polymerization inhibitor means an amount of the polymerization inhibitors effective to prevent premature polymerization during storage. Suitable polymerization inhibitors include a mixture of anionic polymerization inhibitors and free radical polymerization inhibitors.

Mixtures of anionic and free radical polymerization inhibitors are most preferred and, in one embodiment, a mixture of hydroquinone, 4-methoxyphenol, and sulfur dioxide is employed. A preferred version of this polymerization inhibitor blend includes 400 ppm hydroquinone, 400 ppm 4-methoxyphenol, and 150 ppm sulfur dioxide. This particular blend has been found to provide a highly stable cyanoacrylate adhesive when used in conjunction with the phenol antimicrobial agent. In another preferred embodiment, 2000 ppm butylated hydroxyanisol (BHA) and 150 ppm sulfur dioxide are used.

Preferred concentration ranges for anionic polymerization inhibitors, in such mixtures of adhesive composition, range from about 50 ppm to about 500 ppm. Examples of suitable anionic polymerization inhibitors include, but are not limited to, sulfur dioxide, sulfonic acids, sulfuric acid, sulfur trioxide, phosphorous acids, carboxylic acids, picric acid, boron trifluoride, and hydrogen fluoride.

Free radical polymerization inhibitors included in mixtures containing blends with anionic polymerization inhibitors are present in concentrations ranging from about 50 to about 3000 ppm. Examples of suitable free radical polymerization inhibitors include, but are not limited to, hydroquinone, 4-methoxyphenol, butylated hydroxyanisol (BHA), and butylated hydroxy toluene (BHT).

Other anionic polymerization inhibitors are known in the art and may be used in the cyanoacrylate compositions described herein.

Such additional inhibitors include very strong acids that have an aqueous pKₐ of less than 1.0. Suitable very strong acidic inhibitors include, but are not limited to, very strong mineral and/or oxygenated acids. Examples of such very strong acids include, but are not limited to, sulfuric acid (pKₐ −3.0), perchloric acid (PKₐ −5), hydrochloric acid (pKₐ −7.0), hydrobromic acid (pKₐ −9), fluorosulfonic acid (pKₐ <−10), chlorosulfonic acid (pKₐ −10). The amount of very strong acid liquid phase anionic stabilizer to be used can be determined by one of ordinary in the art without undue experimentation.

Such additional inhibitors also include acids with higher pKa than the acids described above and may be provided to more precisely control the cure speed and stability of the adhesive, as well as the molecular weight of the cured adhesive.

These additional inhibitors include those having aqueous pKₐ ionization constants ranging 2 to 8, preferably from 2 to 6, and most preferably from 2 to 5. Examples of such inhibitors include, but are not limited to, phosphoric acid (pKₐ 2.2), organic acids, such as acetic acid (pKₐ 4.8), benzoic acid (pKₐ 4.2), chloroacetic acid (pKₐ 2.9), cyanoacetic acid, and mixtures thereof. Preferably these inhibitors are organic such as acetic acid or benzoic acid.

The Optional Biocompatible Plasticizer

The cyanoacrylate adhesive compositions described in this patent may also optionally contain a biocompatible plasticizer that may be any material that is soluble or dispersible in the cyanoacrylate composition, that increases the flexibility of the resulting polymeric film coating on the skin surface, and that, in the amounts employed, is compatible with the as measured by the lack of moderate to severe skin irritation it is substantially non-irritating (i.e., it is substantially non-irritating). Suitable plasticizers are well known in the art and include those disclosed in U.S. Pat. Nos. 2,784,127 and 4,444,933, which are incorporated herein by reference in their entirety. Specific plasticizers include, by way of example only, tributyl acetyl citrate, trihexyl acetyl citrate, butyl benzyl phthalate, dibutyl phthalate, dioctylphthalate, n-butyryl tri-n-hexyl citrate, diethylene glycol dibenzoate, dimethyl sebacate, triethyl phosphate, tri(2-ethylhexyl)phosphate, tri(p-cresyl)phosphate, glyceryl triacetate, glyceryl tributyrate, diethyl sebacate, dioctyl adipate, isopropyl myristate, butyl stearate, lauric acid, trioctyl trimellitate, dioctyl glutarate, and mixtures thereof, and the like. The particular biocompatible plasticizer employed is not critical and preferred plasticizers include dioctylphthalate, tributyl acetyl citrate, and C₂-C₄-acyl tri-n-hexyl citrates. In some embodiments, suitable plasticizers include polymeric plasticizers, such as polyethylene glycol (PEG) esters and capped PEG esters or ethers, polyester glutarates and polyester adipates.

One particularly preferred class of plasticizers is dialkyl phthalates and, in particular, dioctyl phthalate, all of which are disclosed in U.S. Pat. No. 5,480,935 to Greff, et al. which is incorporated herein by reference in its entirety. Another particularly preferred plasticizer is acetyl citrate that is described in U.S. Pat. Nos. 5,665,817 and 6,191,202, both to Greff, et al., which are incorporated herein by reference in its entirety.

When incorporated in the composition, the optional biocompatible plasticizer is present in an amount effective to increase flexibility of the resulting polymer film and in an amount that is substantially non-irritating. In a preferred embodiment the amount of plasticizer ranges from about 0.5 to about 30, or from about 10 to about 30 weight percent based on the total weight of the composition. In a more preferred embodiment the amount of plasticizer ranges from about 15 to about 25 weight percent based on the total weight of the composition. The plasticizing agent or agents may also be present in amounts ranging from about 0.5 wt. % to about 25 wt. %, or from about 1 wt. % to about 20 wt. % or from about 3 wt. % to about 15 wt. % or from about 5 wt. % to about 7 wt. %. Preferably, the biocompatible plasticizer is included.

Other Components

The polymerizable cyanoacrylate adhesive optionally contains a thickening agent and may additionally contain one or more optional additives such as medicaments, colorants, perfumes, anti-diffusion agents, rubber modifiers, modifying agents, etc. In practice, each of these optional additives should be both miscible and compatible with the cyanoacrylate composition and the resulting polymer. Compatible additives are those that do not prevent the use of the cyanoacrylates in the manner described herein.

The optional thickening agent may be any biocompatible material that increases the viscosity of the composition. Suitable thickening agents include, by way of example, polymethyl methacrylate (PMMA) or other preformed polymers soluble or dispersible in the composition, a suspending agent such as fumed silica and the like with PMMA being preferred. Fumed and modified fumed silica are particularly useful in producing a gel for topical application having a viscosity of from about 1,500 to about 1,000,000 cSt at 20° C. Suitable thickening agents for the compositions described herein also include a partial polymer of the alkyl cyanoacrylate as disclosed in U.S. Pat. Nos. 3,654,239 and 4,038,345 both of which are incorporated herein by reference in their entirety.

Thickening agents are deemed to be biocompatible if they are soluble or dispersible in the composition and are compatible with the skin as measured by the lack of moderate to severe skin irritation.

The concentration of thickening agent employed is preferably an amount sufficient to form a stable suspension or gel with the polymerizable cyanoacrylate ester composition. This, in turn, correlates with the viscosity of the composition. For example, stable suspensions are preferably achieved by addition of sufficient thickening agent to provide for a viscosity of from about 50 to 50,000 centipoise at 20° C. For gel forms, it is preferred to add sufficient thickening agent into the composition to impart a viscosity of from about 1,500 to about 1,000,000 centipoise at 20° C. at zero shear. Preferably, the composition is thixotropic such that application of the composition to the tissue is significantly enhanced.

In a particularly preferred embodiment, the composition will comprise about 0.5 to about 10 percent by weight of the thickening agent based on the total weight of the composition wherein the composition will have a viscosity of about 50 to about 1,000,000 centipoise at 20° C.

In general, colorants are added so that the polymer layer formed on the skin will contain a discrete and discernable color. Perfumes are added to provide a pleasant smell to the formulation. Rubber modifiers are added to further enhance the flexibility of the resulting polymer layer. Medicaments are added as necessary to achieve a desired prophylactic or therapeutic effect. The amount of each of these optional additives employed in the composition is an amount necessary to achieve the desired effect.

Other medicaments suitable for use in conjunction with the cyanoacrylate composition include cortico steroids such as described by Greff, et al. in U.S. Pat. No.5,962,010, incorporated herein by reference in its entirety, and analgesic and/or anesthetic compounds such as lidocaine. The former reduces inflammation at the tissue site whereas the latter reduces pain. Combinations of steroids and analgesics are also suitable.

Specific Formulations:

Specific cyanoacrylate adhesive compositions that may be used include but are not limited to the following:

An n-butyl cyanoacrylate composition: A composition comprising n-butyl cyanoacrylate, phenol, a blend of anionic and free radical inhibitors, and a plasticizer.

In a preferred version of this composition, the amount of phenol is about 0.01% to about 5% of the total weight of the composition. In a more preferred version of this composition, the amount of phenol is about 0.1% to about 1.4%. In a more preferred version the phenol is present in about 0.1%, about 0.6%, or about 1% of the composition.

In another preferred version of this composition, a blend of anionic and free radical inhibitors is employed. In another preferred version the blend of anionic and free radical inhibitors comprises sulfur dioxide, and hydroquinone. In yet another preferred version the blend of anionic and free radical inhibitors comprises sulfur dioxide, 4-methoxyphenol and hydroquinone. In still another preferred version of this composition, the blend of anionic and free radical inhibitors contains a ppm amount of anionic inhibitor(s) and a ppm amount of free radical inhibitor(s) in a ratio of about 1:1 to about 1:8, with ratios of about 1:1 and about 1:4 being more preferred. In a more preferred version of the composition, the blend of anionic and free radical inhibitors contains a ppm amount of sulfur dioxide and a ppm amount of hydroquinone in a ratio of about 1:1 to about 1:4, with 1:1 and 1:4 being the more preferred ratios. In a more preferred version of the composition, the blend of anionic and free radical inhibitors contains a ppm amount of sulfur dioxide and a ppm amount of hydroquinone and a ppm amount of 4-methoxyphenol in a ratio of about 1:1:1 to about 1:4:4 (sulfur dioxide:hydroquinone:4-methoxyphenol) with ratios of about 1:1: and about 1:4:4 being more preferred. In a more preferred version of the composition, the blend of anionic and free radical inhibitors contains either a blend of about 100 ppm of sulfur dioxide and about 400 ppm of hydroquinone or a blend of about 100 ppm of sulfur dioxide and about 100 ppm of hydroquinone. In an even more preferred version of the composition, the blend of anionic and free radical inhibitors contains either a blend of about 100 ppm of sulfur dioxide and about 400 ppm of hydroquinone and 400 ppm of 4-methoxyphenol or a blend of about 100 ppm of sulfur dioxide and about 100 ppm of hydroquinone and 400 ppm of 4-methoxyphenol.

In another preferred version of this composition, the plasticizer is tributyl acetyl citrate or di-ethylhexyl phthalate. In another preferred version of the composition, the weight of plasticizer and weight of n-butyl cyanoacrylate are in a ratio of about 1:3 to about 1:4. In a more preferred version of the composition, the plasticizer is either (1) tributyl acetyl citrate and the weight of plasticizer and weight of n-butyl cyanoacrylate are in a ratio of about 1:4, or (2) di-ethylhexyl phthalate and the weight of plasticizer and weight of n-butyl cyanoacrylate are in a ratio of about 1:3.

In a most preferred version of this composition, the amount of phenol is about 1% of the total weight of the composition, the blend of anionic and free radical inhibitors contains a blend of about 100 ppm of sulfur dioxide and about 400 ppm of hydroquinone and 400 ppm of 4-methoxyphenol, the plasticizer is tributyl acetyl citrate, and the weight of plasticizer and weight of n-butyl cyanoacrylate are in a ratio of about 1:4.

In another most preferred version of this composition, the amount of phenol is about 1% of the total weight of the composition, the blend of anionic and free radical inhibitors contains a blend of about 100 ppm of sulfur dioxide, about 100 ppm of hydroquinone and 400 ppm of 4-methoxyphenol, the plasticizer is di-ethylhexyl phthalate, and the weight of plasticizer and weight of n-butyl cyanoacrylate are in a ratio of about 1:3.

Blend of cyanoacrylate and decyl cyanoacrylate composition: A composition comprising a blend of n-butyl cyanoacrylate and decyl cyanoacrylate, phenol, a blend of anionic and free radical inhibitors, and an optional plasticizer.

In a preferred version of this composition, the weight of n-butyl cyanoacrylate and weight of decyl cyanoacrylate are present in a ratio of about 1:1 to about 3:7.

In a preferred version of this composition, the amount of phenol is about 0.01% to about 5% of the total weight of the composition. In a more preferred version of this composition, the amount of phenol is about 0.1% to about 1.4%. In a more preferred version the phenol is present in about 0.1%, about 0.6%, or about 1% of the composition.

In another preferred version of this composition, the blend of anionic and free radical inhibitors contains a blend of sulfur dioxide and hydroquinone. In another preferred version of this composition, the blend of anionic and free radical inhibitors contains a ppm amount of anionic inhibitor and a ppm amount of free radical inhibitor in a ratio of about 1:1. In a more preferred version of the composition, the blend of anionic and free radical inhibitors contains a ppm amount of sulfur dioxide and a ppm amount of hydroquinone in a ratio of about 1:1. In an even more preferred version of the composition, the blend of anionic and free radical inhibitors contains a blend of about 100 ppm of sulfur dioxide and about 100 ppm of hydroquinone.

In another preferred version of this composition, the optional plasticizer is absent.

In a most preferred version of this composition, the weight of n-butyl cyanoacrylate and weight of decyl cyanoacrylate are in a ratio of about 3:7, the amount of phenol is about 0.1% or about 0.6% of the total weight of the composition, the blend of anionic and free radical inhibitors contains a blend of about 100 ppm of sulfur dioxide and about 100 ppm of hydroquinone, and the plasticizer is absent.

Blend of decyl cyanoacrylate and ethoxyethyl cyanoacrylate composition: A composition comprising a blend of decyl cyanoacrylate and ethoxyethyl cyanoacrylate, phenol, a blend of anionic and free radical inhibitors, and an optional plasticizer.

In a preferred version of this composition, the weight of decyl cyanoacrylate and weight of ethoxyethyl cyanoacrylate are in a ratio of about 1:1.

In a preferred version of this composition, the amount of phenol is about 0.01% to about 5% of the total weight of the composition. In a more preferred version of this composition, the amount of phenol is about 0.1% to about 1.4%. In a more preferred version the phenol is present in about 0.1%, about 0.6%, or about 1% of the composition.

In another preferred version of this composition, the blend of anionic and free radical inhibitors contains a blend of sulfur dioxide and hydroquinone. In another preferred version of this composition, the blend of anionic and free radical inhibitors contains a ppm amount of anionic inhibitor and a ppm amount of free radical inhibitor in a ratio of about 1:1. In a more preferred version of the composition, the blend of anionic and free radical inhibitors contains a ppm amount of sulfur dioxide and a ppm amount of hydroquinone in a ratio of about 1:1. In an even more preferred version of the composition, the blend of anionic and free radical inhibitors contains a blend of about 100 ppm of sulfur dioxide and about 100 ppm of hydroquinone.

In another preferred version of this composition, the optional plasticizer is absent.

In a most preferred version of this composition, the weight of decyl cyanoacrylate and weight of ethoxyethyl cyanoacrylate are in a ratio of about 1:1, the amount of phenol is about 0.1% or about 0.6% of the total weight of the composition, the blend of anionic and free radical inhibitors contains a blend of about 100 ppm of sulfur dioxide and about 100 ppm of hydroquinone, and the plasticizer is absent.

Blend of n-butyl cyanoacrylate and octyl cyanoacrylate composition: A composition comprising a blend of n-butyl cyanoacrylate and octyl cyanoacrylate, phenol, a blend of anionic and free radical inhibitors, and an optional plasticizer.

In a preferred version of this composition, the weight of n-butyl cyanoacrylate and weight of octyl cyanoacrylate are present in a ratio of about 1:1 to about 3:7. In another embodiment the weight of n-butyl cyanoacrylate and weight of octyl cyanoacrylate are present in a ratio of about 56:30.

In a preferred version of this composition, the amount of phenol is about 0.01% to about 5% of the total weight of the composition. In a more preferred version of this composition, the amount of phenol is about 0.1% to about 1.4%. In a more preferred version the phenol is present in about 0.1%, about 0.6%, or about 1% of the composition.

In another preferred version of this composition, the blend of anionic and free radical inhibitors contains a blend of sulfur dioxide and hydroquinone. In another preferred version of this composition, the blend of anionic and free radical inhibitors contains a ppm amount of anionic inhibitor and a ppm amount of free radical inhibitor in a ratio of about 1:1. In a more preferred version of the composition, the blend of anionic and free radical inhibitors contains a ppm amount of sulfur dioxide and a ppm amount of hydroquinone in a ratio of about 1:1. In an even more preferred version of the composition, the blend of anionic and free radical inhibitors contains a blend of about 100 ppm of sulfur dioxide and about 100 ppm of hydroquinone.

In another preferred version of this composition, the optional plasticizer is present in about 14 weight percent.

Methods of Use

The methods of this invention comprise the in situ formation of an antimicrobial, cyanoacrylate polymer film on mammalian tissue such as mammalian skin.

The treatment protocol preferably involves tissue preparation prior to in situ formation of the cyanoacrylate polymer. For example, mammalian skin is first conventionally treated by cleaning with an appropriate antimicrobial composition. The skin is preferably dried, e.g., blotted dry, and then an adherent antimicrobial polymeric film is formed over this site by applying a cyanoacrylate composition of this invention. As noted above, this composition comprises polymerizable cyanoacrylate monomers and/or reactive oligomers that, upon contact with the skin, polymerize in situ to form a polymeric film. Pretreatment of other mammalian tissue will also follow conventional procedures.

Polymerization occurs at ambient conditions for a sufficient period of time to allow robust films to form. In general, the particular length of time required for polymerization will vary depending on factors such as the amount of adhesive composition applied, the temperature of the tissue, the moisture content of the tissue, the surface area of tissue, and the like. However, in a preferred embodiment, polymerization is generally complete within about 10 to about 60 seconds while the tissue is maintained at ambient conditions; however, in some cases, polymerization can occur in as little as 5 seconds and up to about 5 minutes. During this period, the tissue is maintained in a position that permits the cyanoacrylate to polymerize and form a polymeric film while minimizing any movement that might dislodge the cyanoacrylate from the tissue or create undesirable bonding.

Sufficient amounts of the composition are employed to cover (i.e., coat) the entire tissue site with a layer of the cyanoacrylate polymer. If necessary, excess cyanoacrylate monomer and/or oligomer can be removed with a wipe or tissue paper before polymerization or, after polymerization, any polymer formed at unintended sites can be removed with materials such as acetone.

After polymerization, the resulting polymeric film forms an antimicrobial, barrier film that strongly adheres to the skin and is both flexible and waterproof. Such strong adherence effectively eliminates the possibility that the film will separate from the tissue. In the case of application to mammalian skin, the polymeric film will only adhere to the skin for a period of about 1-4 days after which time it sloughs off. This occurs because the cyanoacrylate polymer is adhering only to the epidermal layer that is continuously in the process of being sloughed off and replaced by the underlying cells. Accordingly, the cyanoacrylate film need not be removed from such skin.

The polymeric film should be maintained in an unbroken manner over the entire tissue. This can be assured by careful application of the cyanoacrylate adhesive onto the tissue. Additionally, the use of the optional biocompatible plasticizer will facilitate the maintenance of the polymeric film in an unbroken manner and will inhibit cracking of the film.

In one embodiment, after application of the initial polymeric layer, a second, preferably thinner, layer is applied thereto. Additional amounts of cyanoacrylate composition can be applied as needed to maintain an unbroken coating covering over the tissue.

When the preferred mixture of n-butyl cyanoacrylate and octyl cyanoacrylate esters or the alternative preferred mixture of n-butyl cyanoacrylate and decyl cyanoacrylate esters are employed, the resulting polymeric film evidences improved durability in maintaining an unbroken film over the skin over time. In some cases, an unbroken film can be maintained on the skin up to 7 days after application without the need for reapplication. Similar results have been observed with the also preferred mixture of n-butyl cyanoacrylate and decyl cyanoacrylate.

Application is conducted under conditions wherein the polymeric film preferably has a thickness of no more than about 1 millimeter and, more preferably, the polymer layer has a thickness of from about 2 to about 500 microns and still more preferably from about 20 to about 100 microns. If thinner polymeric films are desired, then the polymeric film should have a thickness of from about 2 to about 50 microns and preferably from 10 to 40 microns. The amount of cyanoacrylate composition applied to a unit area to obtain such thicknesses is well within the skill of the art.

The size and thickness of the polymeric film formed onto the tissue area can be readily controlled by the amount and viscosity of cyanoacrylate adhesive composition packaged in a single dose product or by use of a multiple use dispenser that governs the amount of material applied onto a unit area of surface skin. In this regard, the dispenser described by Otake, U.S. Pat. No. 4,958,748, which is incorporated by reference in its entirety, is one example of a dispenser that dispenses the cyanoacrylate adhesive composition in a controlled dropwise manner. Other methods for the controlled dispersement of the cyanoacrylate adhesive include, by way of example, a spray applicator, brush, wipe, swab or solid paddle applicator, applicators for repeated and intermittent use of the cyanoacrylate composition and the like.

In applicators, the cyanoacrylate composition is stored at ambient conditions and can be provided in sterile form.

Method of Preparation

The cyanoacrylate adhesive compositions can be prepared by conventional methods of mixing the appropriate components until homogenous. The adhesive compositions are generally prepared by mixing sufficient amounts of the various components, to achieve the effective concentration.

Generally, the viscosity of the composition is controlled by the nature and concentration of the cyanoacrylate ester, the presence and nature of the optional thickening agent or by the amount of any other material employed. For example, high viscosity compositions that employ lower concentrations of cyanoacrylate esters can be achieved by use of very high molecular weight oligomers. Such factors are well known in the art.

The concentration of polymerizable cyanoacrylate ester employed in the composition is preferably from about 50 to about 99.5 percent by weight based on the total weight of the composition, and more preferably from about 75 to about 89.5 percent by weight.

Still further, the concentration of phenol employed in the composition is sufficient to render the resulting polymeric film antimicrobial. That is to say that the concentration of phenol in the film is sufficient to ensure that the film exhibits antimicrobial activity when measured by conventional assays such as that described in Greff, et al.⁹

If desired, the cyanoacrylate adhesive composition can be degassed. The degassing can be performed by any conventional degassing technique, i.e., thermal or vacuum treatment.

The resulting cyanoacrylate adhesive composition is optionally heat-sterilized and then stored, preferably in sealed vials until needed. In still another preferred embodiment, the cyanoacrylate adhesive composition is sterilized by dry heating the composition under conditions sufficient to sterilize the composition. Other sterilization techniques can be used including, for example, electron beam sterilization, visible light irradiation, and the like. See, for example, U.S. Pat. No. 6,248,800 which is incorporated herein by reference in its entirety.

Utility

The methods described herein are useful in forming an antimicrobial polymeric film over mammalian tissue. This polymeric film finds particular utility in treating an existing bacterial infection at the tissue site or in inhibiting this tissue from becoming infected with bacteria. Suitable mammals for use in these methods preferably include humans as well as domestic animals such as horses, cows, dogs, sheep, cats, etc.

The following examples illustrate certain embodiments of the invention but are not intended to limit the scope of the claims in any way. Unless otherwise indicated, all temperatures are in degrees Celsius, all percents are weight percents based on the entire weight of the composition. In addition, the following abbreviations have the following meanings:
cP=centipoises
hr=hours
min=minute
ppm=ppm
PSI=pounds per square inch
sec=seconds
w/w=weight/weight
   Undefined terms have their art recognized meanings.

Examples 1-4 below illustrate the advantageous features of the invention and Examples 5-7 illustrate how the methods of this invention could be practiced.

### EXAMPLE 1

Seven antimicrobial agents taken from the FDA draft monograph for First Aid Products were evaluated for their compatibility with cyanoacrylate adhesives. The evaluation consisted of mixing the liquid or powdered solid at the recommended concentration directly into an ethyl cyanoacrylate composition and observing the reaction over 7 days. The ethyl cyanoacrylate composition was purchased from Parson International (Rochester, Mich., USA) as Parfix 105 low viscosity and contains proprietary inhibitors that are believed to be hydroquinone and either sulfur dioxide or methane sulfonic acid. No other additional components were added except the antimicrobial agents. The tests were conducted at room temperature. If the mixture was stable for 7 days the set time of the mixture on skin was determined to ensure the mixture was capable of setting.

TABLE 1

| Ethyl | | | |
|---|---|---|---|
| Cyanoacrylate | | | |
| and | | | |
| Antimicrobial | Observations | OK @ 7 days | Set time |
| 10% povidone | Settles out and | NO | — |
| iodine | sets in ˜1-2 day | | |
| 10% povidone | Stable when | YES | 25 sec |
| iodine stirred | stirred for >7 days | | |
| 0.1% | Immediate exothermic | NO | — |
| Benzalkonium | polymerization | | |
| chloride | around powder, | | |
| | complete set 1 hour | | |
| 0.15% | Immediate exothermic | NO | — |
| Benzethonium | polymerization | | |
| Chloride | around powder, | | |
| | complete set 1 hour | | |
| 0.1% Hexyl | Stable >7 days | YES | ˜15 sec |
| resorcinol | | | |
| 1.0% phenol | Stable >7 days | YES | ˜15 sec |
| 3% camphor | Stable >7 days | YES | ˜15 sec |
| 1% m-cresol | | | |
| 27% | Exothermic set | NO | — |
| ethanol/essential | 10-15 minutes | | |
| oils mix* | | | |

*consists of 26.9% ethanol, 0.063% thymol, 0.042% menthol, 0.055% methyl salicylate,0.001% eucalyptol

### EXAMPLE 2

Three antimicrobial agents from Example 1, which are apparently compatible with cyanoacrylate adhesives, were tested to determine their aging properties when mixed in their “effective antimicrobial” concentrations in a composition containing 80% butyl cyanoacrylate, 20% tributyl acetyl citrate, and approximately 100 ppm of sulfur dioxide, 400 ppm of 4-methoxyphenol and 400 ppm of hydroquinone, and stored at 60° C. As used in this example and example 3 only, the “effective antimicrobial” concentration means that amount determined using the FDA draft monograph for First Aid Products as having antimicrobial activity.

TABLE 2

| | | | |
|---|---|---|---|
| | 0.1% hexyl | | 4% camphor/m- |
| Test | resorcinol | 1% phenol | cresol |

Time zero

| | | | |
|---|---|---|---|
| Viscosity cP | 2.9 | 4.5 | 4.9 |
| Adhesive strength | 110% | 172% | 120% |
| 10 min* | | | |
| Adhesive strength | 200% | 376% | 188% |
| 24 hr* | | | |
| Set time salt | 8 | 6 | 6 |
| solution (sec) | | | |
| Set time skin (sec) | 20 | 17 | 17 |
| Wear time | 3 days | 3 days | 2.5 days |
| Visual | OK | OK | OK |

14 days @ 60° C.

| | | | |
|---|---|---|---|
| Viscosity cP | 6.4 | 4.4 | 4.4 |
| Adhesive strength | 29% | 100% | 92% |
| 10 min* | | | |
| Adhesive 24 hr* | 60% | 128% | 96% |
| Set time salt | 33 | 23 | 25 |
| solution (sec) | | | |
| Set time skin (sec) | 27 | 12 | 15 |
| Wear time | 2 days | 4 days | 2.5 days |
| Visual | Yellow | OK | OK |

77 days @ 60° C.

| | | | |
|---|---|---|---|
| Viscosity cP | Solid | 7.0 | 7.3 |
| Adhesive strength | Solid | 190% | 140% |
| 10 min* | | | |
| Adhesive 24 hr* | Solid | 248% | 144% |
| Set time salt | Solid | 7 | 18 |
| solution (sec) | | | |
| Set time skin (sec) | Solid | 12 | 30 |
| Wear time | Solid | 3 days | 1 day |
| Visual | Solid | Slight beige tint | Brown |

*Expressed relative to strength required for effective wound closure

The above data demonstrates that the use of phenol provides unexpected enhancement in adhesive strength in the resulting polymeric film as compared to other antimicrobials employed.

### EXAMPLE 3

Three antimicrobial agents from Example 1, which are apparently compatible with cyanoacrylate adhesives, were tested to determine their aging properties when mixed in their effective antimicrobial concentrations in a composition containing 80% butyl cyanoacrylate, 20% tributyl acetyl citrate, and approximately 100 ppm of sulfur dioxide and 400 ppm of hydroquinone, and stored under accelerated shelf-life conditions at 82° C. As before, the effective antimicrobial concentration was determined using the FDA draft monograph for First Aid Products.

TABLE 3

| | Viscosity/Color | | |
|---|---|---|---|
| | 0.1% hexyl | | |
| Time | resorcinol | 1% phenol | 4% camphor/m-cresol |
| Time zero | Low/clear | Low/clear | Low/clear |
| 3 days | Low/light yellow | Low/clear | Low/clear |
| 14 days | Low/yellow | Low/pale beige | Low/pale beige |
| 28 days | Med/yellow | Low/pale beige | Low-med/pale beige |
| 55 days | Solid/yellow | Low-med/beige | Med-high/light brown |

In this test, stability is measured by the lack of color change and/or solid formation. In particular, instability of the composition results in degradation and/or polymerization under accelerated shelf-life conditions. This, in turn, manifests itself by color change with the more intense color change reflecting greater instability and solidification (the latter being the end point in lack of stability).

The above data demonstrates a significant increase in stability of the composition comprising 1% phenol as compared to the other two cyanoacrylate compositions comprising other antimicrobial agents in their recommended amounts.

### EXAMPLE 4

In this example, three different polymerizable cyanoacrylate ester blends were tested. The blends used were (1) 30:70 w/w butyl cyanoacrylate: decyl cyanoacrylate, containing 100 ppm sulfur dioxide and 100 ppm hydroquinone; (2) 50:50 w/w decyl cyanoacrylate: ethoxyethyl cyanoacrylate containing 100 ppm sulfur dioxide and 100 ppm hydroquinone; and (3) 75:25 w/w butyl cyanoacrylate: di-ethylhexyl phthalate containing 100 ppm sulfur dioxide and 100 ppm hydroquinone. Each of these blends was tested with 0.1% hexyl resorcinol, 0.1% phenol, and 0.6% phenol, as the added anti-microbial agent. The first two tested compositions did not contain any plasticizer. All tested compositions were prepared by simple mixing of the components.

All compositions were tested at time zero (when manufactured), and then aged under the following conditions: (1) 3 days at 82° C.; (2) 2 weeks at room temperature; (3) 2 weeks at 60° C.; and (4) 2 weeks at 82° C. For each composition and for each aging regime, the following testes were conducted, the results of which are provided in table 4 below: (1) Setting time using Earle's balanced salts in seconds; (2) Setting time on skin; (3) Viscosity in centipoises (cP) using a Brookfield viscometer; and (4) adhesive bond strength using butt joints in neoprene rubber tested after 10 minutes and 24 hours, results expressed in pounds per square inch.

Detailed below are the stability data for cyanoacrylate formulations containing phenol as compared to another less preferred antimicrobial agent, such as hexyl resorcinol. The shelf-life of adhesive compositions can be limited without the presence of phenol. The viscosity tends to increase and the solution will turn yellow and/or gray as the solution ages. The shelf-life of a given composition can be approximated by measuring the viscosity, adhesion and set time of the adhesive compositions after storing the composition at elevated temperatures. The data shown in Table 4-6 below show that the presence of phenol in either 0.1% or 0.6% increases the stability of polymerizable cyanoacrylate monomer compositions relative to compositions containing hexyl resorcinol as determined using viscosity and visual appearance as a metric. For all the cyanoacrylate compositions tested, the viscosity of the phenol containing compositions after 14 days at about 82° C. (which are conditions sufficient to estimate about 2 to 3 years of shelf storage) were at least 50% less viscous than the control solution. The adhesion and the set times of all the measured compositions have remained within acceptable and useful ranges.

TABLE 4

| | | | |
|---|---|---|---|
| | 0.1% | | |
| | hexyl | 0.1% | 0.6% |
| Test | resorcinol | phenol | phenol |

Time zero

| | | | |
|---|---|---|---|
| Viscosity cP | 6.5 | 6.2 | 6.4 |
| Adhesion 10 min (PSI) | 9.6 | 14.1 | 22.1 |
| Adhesion 24 hr (PSI) | 8.2 | 14.1 | 14.4 |
| Set time Earle's (sec) | 124 | 136 | 136 |
| Set time skin (sec) | 14 | 19 | 15 |
| Visual | OK | OK | OK |

2 weeks room temperature

| | | | |
|---|---|---|---|
| Viscosity cP | 6.4 | 6.2 | 6.4 |
| Adhesion 10 min (PSI) | 8.0 | 8.0 | 11.4 |
| Adhesion 24 hr (PSI) | 10.6 | 6.7 | 13.0 |
| Set time Earle's (sec) | 117 | 121 | 132 |
| Set time skin (sec) | 13 | 8.5 | 9.5 |
| Visual | OK | OK | OK |

14 days 60° C.

| | | | |
|---|---|---|---|
| Viscosity cP | 6.8 | 6.6 | 6.6 |
| Adhesion 10 min (PSI) | 0.8 | 7.4 | 10.0 |
| Adhesion 24 hr (PSI) | 4.3 | 6.4 | 5.1 |
| Set time Earle's (sec) | 109 | 145 | 160 |
| Set time skin (sec) | 11.5 | 13.5 | 10 |
| Visual | Pale | v. slight | v. slight |
| | yellow | gray | gray |

3 days 82° C.

| | | | |
|---|---|---|---|
| Viscosity cP | 7.3 | 6.9 | 6.4 |
| Adhesion 10 min (PSI) | 19.0 | 12.3 | 16.6 |
| Adhesion 24 hr (PSI) | 10.2 | 9.1 | 14.4 |
| Set time Earle's (sec) | 96 | 76 | 132 |
| Set time skin (sec) | 8 | 13 | 10 |
| Visual | yellow | Slight | Slight |
| | | yellow/gray | yellow/gray |

14 days 82° C.

| | | | |
|---|---|---|---|
| Viscosity cP | 202.1 | 29.5 | 25.6 |
| Adhesion 10 min (PSI) | 79.2 | 71.4 | 60.2 |
| Adhesion 24 hr (PSI) | 66.2 | 59.5 | 35.7 |
| Set time Earle's (sec) | 2 | 3 | 1 |
| Set time skin (sec) | 6 | 11 | 8 |
| Visual | Strong | Pale | Pale |
| | yellow | yellow/gray | yellow/gray |

70:30 decyl cyanoacrylate:butyl cyanoacrylate;
100 ppm SO₂;
100 ppm hydroquinone

TABLE 5

| | | | |
|---|---|---|---|
| | 0.1% hexyl | | |
| Test | resorcinol | 0.1% phenol | 0.6% phenol |

Time zero

| | | | |
|---|---|---|---|
| Viscosity cP | 7.6 | 7.1 | 7.3 |
| Adhesion 10 min (PSI) | 106 | 92 | 82 |
| Adhesion 24 hr (PSI) | 67 | 76 | 66 |
| Set time Earle's (sec) | 1.5 | 2 | 2 |
| Set time skin (sec) | 6 | 5 | 6.5 |
| Visual | OK | OK | OK |

2 weeks room temperature

| | | | |
|---|---|---|---|
| Viscosity cP | 7.6 | 7.5 | 7.2 |
| Adhesion 10 min (PSI) | 63 | 50 | 47 |
| Adhesion 24 hr (PSI) | 43.0 | 43.4 | 59.8 |
| Set time Earle's (sec) | 1 | 2 | 1 |
| Set time skin (sec) | 5 | 4.5 | 5.5 |
| Visual | OK | OK | OK |

14 days 60° C.

| | | | |
|---|---|---|---|
| Viscosity cP | 7.8 | 6.9 | 7.5 |
| Adhesion 10 min (PSI) | 51 | 41.6 | 50 |
| Adhesion 24 hr (PSI) | 23.5 | 23.5 | 33.8 |
| Set time Earle's (sec) | 3.0 | 4.5 | 3.5 |
| Set time skin (sec) | 6 | 6.5 | 5 |
| Visual | v. slight yellow | OK | OK |

3 days 82° C.

| | | | |
|---|---|---|---|
| Viscosity cP | 7.8 | 7.0 | 7.4 |
| Adhesion 10 min (PSI) | 39.0 | 39.7 | 49.1 |
| Adhesion 24 hr (PSI) | 60 | 68 | 51 |
| Set time Earle's (sec) | 2 | 4 | 2.5 |
| Set time skin (sec) | 4.5 | 8.5 | 7.5 |
| Visual | Slight yellow | OK | OK |

14 days 82° C.

| | | | |
|---|---|---|---|
| Viscosity cP | 29.2 | 13.7 | 13.0 |
| Adhesion 10 min (PSI) | 69.6 | 57.0 | 77.6 |
| Adhesive 24 hr (PSI) | 70.6 | 55.7 | 56.8 |
| Set time Earle's (sec) | 2 | 4 | 3 |
| Set time skin (sec) | 6.5 | 10 | 4.5 |
| Visual | Pale yellow | Slight yellow | Slight yellow |

50:50 decyl cyanoacrylate:ethoxyethyl cyanoacrylate;
100 ppm SO₂;
100 ppm hydroquinone

TABLE 6

| | | | |
|---|---|---|---|
| | 0.1% hexyl | | |
| Test | resorcinol | 0.1% phenol | 0.6% phenol |

Time zero

| | | | |
|---|---|---|---|
| Viscosity cP | 4.6 | 4.3 | 4.6 |
| Adhesion 10 min (PSI) | 29.4 | 21.2 | 26.4 |
| Adhesion 24 hr (PSI) | 26.1 | 30.2 | 39.2 |
| Set time Earle's (sec) | 8 | 8 | 9 |
| Set time skin (sec) | 3.5 | 2.5 | 3 |
| Visual | OK | OK | OK |

2 weeks room temperature

| | | | |
|---|---|---|---|
| Viscosity cP | 4.9 | 4.4 | 4.7 |
| Adhesion 10 min (PSI) | 17.0 | 13.8 | 13.0 |
| Adhesion 24 hr (PSI) | 20.6 | 17.6 | 31.0 |
| Set time Earle's (sec) | 11 | 11 | 12 |
| Set time skin (sec) | 5.5 | 7 | 10 |
| Visual | OK | OK | OK |

14 days 60° C.

| | | | |
|---|---|---|---|
| Viscosity cP | 5.0 | 4.4 | 4.8 |
| Adhesion 10 min (PSI) | 17.3 | 14.1 | 25.8 |
| Adhesion 24 hr (PSI) | 17.1 | 23.5 | 26.6 |
| Set time Earle's (sec) | 46 | 26 | 27 |
| Set time skin (sec) | 7 | 9 | 10 |
| Visual | Slight yellow | OK | OK |

3 days 82° C.

| | | | |
|---|---|---|---|
| Viscosity cP | 4.9 | 5.2 | 4.9 |
| Adhesion 10 min (PSI) | 13.3 | 18.1 | 15.8 |
| Adhesion 24 hr (PSI) | 24 | 15.2 | 28.5 |
| Set time Earle's (sec) | 27 | 18 | 21 |
| Set time skin (sec) | 8 | 7 | 8 |
| Visual | v. slight yellow | OK | OK |

14 days 82° C.

| | | | |
|---|---|---|---|
| Viscosity cP | 12.7 | 6.0 | 6.2 |
| Adhesion 10 min (PSI) | 33.0 | 20.2 | 19.7 |
| Adhesion 24 hr (PSI) | 43.2 | 29.6 | 23.0 |
| Set time Earle's (sec) | 22 | 37 | 40 |
| Set time skin (sec) | 5.5 | 7 | 4 |
| Visual | Slight yellow | v. slight yellow | v. slight yellow |

75:25 butyl cyanoacrylate:di-ethylhexyl phthalate (DOP),
100 ppm SO₂;
100 ppm hydroquinone

### EXAMPLE 5

A man subject to recurrent skin infections presents himself to the attending clinician with a large area of the inner right thigh that is beginning to redden and itch. The involved area and the surrounding two inches of skin are coated with a low viscosity liquid composition comprising 56% n-butyl cyanoacrylate, 30% octyl cyanoacrylate, 14% tributyl acetyl citrate plasticizer (all percentages are weight percentages based on the total weight of the composition). Subsequently 5000 ppm phenol, 400 ppm hydroquinone, 400 ppm 4-methoxyphenol, and 150 ppm sulfur dioxide are added this mixture. The composition spreads easily and sets within 60 seconds to form a coherent flexible polymeric film.

### EXAMPLE 6

A 13-year-old boy playing with ointments from the medicine cabinet causes a chemical bum on his left forearm by miss-using a depilatory cream. The approximately 2-inch diameter area is painful, red and swollen and the outer layer of skin is starting to peel off. The boy's mother thoroughly washes the skin and applies a film of antiseptic liquid bandage made from 80% n-butyl cyanoacrylate, 20% tributyl acetyl citrate plasticizer (all percentages are weight percentages based on the total weight of the composition). Subsequently 0.5% by weight phenol, 150 ppm sulfur dioxide and 500 ppm hydroquinone are added to this mixture.

The liquid is applied as a thin film using a brush applicator in the bottle and sets within 30 seconds with only a mild stinging sensation. Once set, the pain of the burn is largely alleviated and the skin heals within 7 days, at which time the plastic film sloughs off.

### EXAMPLE 7

An experienced cyclist loses traction and falls from his bike onto a wooden support while traversing a wet wooden bridge in a park. The fall leaves him with an extensive painful graze on his upper left arm. Because the park bridge is over a swampy area of land and is traversed by many domestic animals he is concerned about the risk of infection in the 6-inch by 3-inch abrasion. He covers the abrasion with an antiseptic liquid bandage made from 60% n-butyl cyanoacrylate, 40% n-decyl cyanoacrylate (all percentages are weight percentages based on the total weight of the composition). Subsequently 1% phenol, 1000 ppm butylated hydroxyanisol and 150 ppm sulfur dioxide are added to this mixture. The film sets in 20 seconds giving almost immediate relief from most of the pain, and stays in place until the abrasion heals 6 days later.

From the foregoing description, various and changes in the composition and method will occur to those skilled in the art. All such modifications coming within the scope of the appended claims are intended to be included therein.

1. A cyanoacrylate adhesive composition comprising:
(a) a polymerizable cyanoacrylate ester component;
(b) an antimicrobially effective amount of phenol;
(c) an polymerization inhibiting amount of polymerization inhibitors, and
(d) an optional biocompatible plasticizer.

2. The composition of claim 1, wherein the polymerizable cyanoacrylate ester component comprises a blend of at least two different polymerizable cyanoacrylate esters.

3. The composition of claim 2, wherein the polymerizable cyanoacrylate ester component comprises a blend of n-butyl cyanoacrylate ester and octyl cyanoacrylate ester.

4. The composition of claim 3, wherein the ratio of n-butyl cyanoacrylate ester to octyl cyanoacrylate ester ranges from about 1.5:1 to about 2:1.

5. The composition of claim 2, wherein the polymerizable cyanoacrylate ester component comprises a blend of n-butyl cyanoacrylate ester and decyl cyanoacrylate ester.

6. The composition of claim 5, wherein the ratio of n-butyl cyanoacrylate ester to decyl cyanoacrylate ester ranges from about 1:1 to about 2:1.

7. The composition of claim 1, wherein the polymerizable cyanoacrylate ester component comprises a blend of polymerization inhibitors.

8. The composition of claim 7, wherein the polymerizable cyanoacrylate ester component comprises a blend of hydroquinone, 4-methoxyphenol, and sulfur dioxide.

9. The composition of claim 7, wherein the polymerizable cyanoacrylate ester component comprises a blend of 4-methoxyphenol, butylated hydroxyanisol and sulfur dioxide.

10. The composition of claim 1, wherein the polymerizable cyanoacrylate ester component comprises a cyanoacrylate ester represented by I: 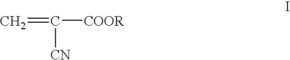
where R is selected from the group consisting of:
alkyl of 1 to 10 carbon atoms,
alkenyl of 2 to 10 carbon atoms,
cycloalkyl groups of from 5 to 8 carbon atoms,
phenyl,
2-ethoxyethyl,
3 -methoxybutyl,
and a substituent of the formula: 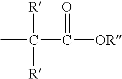
wherein each R′ is independently selected from the group consisting of:
hydrogen and methyl, and
R″ is selected from the group consisting of:
alkyl of 1 to 6 carbon atoms,
alkenyl of from 2 to 6 carbon atoms,
alkynyl of from 2 to 6 carbon atoms,
cycloalkyl of 3 to 8 carbon atoms,
aralkyl selected from the group consisting of benzyl, methylbenzyl and phenylethyl,
phenyl, and
phenyl substituted with 1 to 3 substituents selected from the group consisting of hydroxy, chloro, bromo, nitro, alkyl of 1 to 4 carbon atoms, and alkoxy of from 1 to 4 carbon atoms.

11. The composition of claim 10, wherein R is alkyl of from 2 to 10 carbon atoms.

12. The composition of claim 1 1, wherein R is alkyl of from 2 to 8 carbon atoms.

13. The composition of claim 12, wherein R is selected from the group consisting of butyl, pentyl, octyl, or decyl.

14. The composition of claim 13, wherein R is n-butyl.

15. The composition of claim 13, wherein R is octyl.

16. The composition of claim 13, wherein R is decyl.

17. The composition of claim 1, wherein the optional biocompatible plasticizer is present.

18. The composition of claim 17, wherein the biocompatible plasticizer is present in a concentration ranging from about 10 to about 30 weight percent based on the total weight of the composition.

19. The composition of claim 18, wherein the biocompatible plasticizer is present in a concentration ranging from about 15 to about 25 weight percent based on the total weight of the composition.

20. The composition of claim 18, wherein the biocompatible plasticizer is tributyl acetyl citrate.

21. The composition of claim 1, wherein said cyanoacrylate adhesive composition further comprises a biocompatible thickening agent.

22. A method for the treatment or prevention of infections on mammalian tissues which method comprises:
applying to bacterially infected mammalian tissue or tissue at risk of bacterial infection a cyanoacrylate adhesive composition comprising:
(i) about 50 to about 99.5 weight percent of a polymerizable cyanoacrylate ester based on the total weight of the composition;
(ii) about 0.01 to about 1.4 weight percent phenol based on the total weight of the composition;
(iii) about 100 to about 3,500 ppm of polymerization inhibitors, and
(iv) optionally, about 10 to about 20 weight percent of a biocompatible plasticizer;
in an amount sufficient to form an adherent polymeric film to the tissue where the composition was applied upon polymerization of the cyanoacrylate adhesive composition.

23. The composition of claim 1, wherein the phenol is present in from about 0.01 to about 5 weight percent based on the total weight of the composition.

24. The composition of claim 1, wherein the phenol is present in from about 0.1 to about 1.4 weight percent based on the total weight of the composition.

25. The composition of claim 1, wherein the phenol is present in from about 0.1 to about 1.0 weight percent based on the total weight of the composition.

26. The composition of claim 1, wherein the phenol is present in from about 0.1 to about 0.6 weight percent based on the total weight of the composition.

27. The composition of claim 1, wherein the phenol is present in from about 0.5 to about 1.0 weight percent based on the total weight of the composition.

28. The composition of claim 1, wherein the polymerizable cyanoacrylate ester component is n-butyl cyanoacrylate ester.

29. The composition of claim 1, wherein the polymerizable cyanoacrylate ester component is a blend of n-butyl cyanoacrylate ester and octyl cyanoacrylate ester in a ratio of from about 1.5:1 to about 2:1, respectively.

30. The composition of claim 1, wherein the polymerizable cyanoacrylate ester component is a blend of n-butyl cyanoacrylate ester and decyl cyanoacrylate ester in a ratio of from about 1:1 to about 2:1, respectively.

31. The composition of claim 1, wherein the polymerizable cyanoacrylate ester component is a blend of ethoxyethyl cyanoacrylate ester and decyl cyanoacrylate ester in a ratio of from about 1:1 to about 2:1, respectively.

32. The composition of claim 1, wherein the polymerization inhibitor is selected from the group consisting of a blend of sulfur dioxide and BHA in a ratio of about 1:10 to about 1:15, respectively, a blend of sulfur dioxide and hydroquinone in a ratio of about 1:1 to about 1:4, respectively, and a blend of sulfur dioxide, hydroquinone and 4-methoxyphenol in a ratio of about 1:1:1 to about 3:3:1 or 1:1:1 to about 1.5:4:4, respectively.

33. The composition of claim 1, wherein the plasticizer is a dialkyl phthalate, triakyl acetyl phthalate or tributyl acetyl citrate and is present from about 10 to about 30 percent by weight of the composition.

34. A cyanoacrylate adhesive composition comprising:
(a) a polymerizable cyanoacrylate ester component comprising n-butyl cyanoacrylate ester, a blend of n-butyl cyanoacrylate ester and octyl cyanoacrylate ester, a blend of ethoxyethyl cyanoacrylate ester and decyl cyanoacrylate ester, or a blend of n-butyl cyanoacrylate ester and decyl cyanoacrylate ester;
(b) about 0.1 to about 1.0 weight percent based on total weight of the composition of phenol;
(c) from about 100 ppm to about 3,500 ppm of a polymerization inhibitor selected from the group consisting of a blend of sulfur dioxide and BHA, and a blend of sulfur dioxide and hydroquinone, and a blend of sulfur dioxide, hydroquinone and 4-methoxyphenol; and
(d) an optional biocompatible plasticizer selected from the group consisting of dialkyl phthalate, trialkyl acetyl phthalate and tributyl acetyl citrate.

35. The cyanoacrylate adhesive composition of claim 34, wherein the composition comprises:
(a) n-butyl cyanoacrylate ester;
(b) 0.1 to 1.0 weight percent of phenol;
(c) a blend of sulfur dioxide and hydroquinone; and
(d) a plasticizer.

36. The cyanoacrylate adhesive composition of claim 35, wherein the composition comprises:
(a) 80 weight percent of n-butyl cyanoacrylate ester;
(b) 0.1 to 1.0 weight percent of phenol;
(c) 100 ppm of sulfur dioxide and 100 ppm of hydroquinone; and
(d) the plasticizer is 20 weight percent of tributyl acetyl citrate.

37. The cyanoacrylate adhesive composition of claim 35, wherein the composition comprises:
(a) 75 weight percent of n-butyl cyanoacrylate ester;
(b) 0.1 to 1.0 weight percent of phenol;
(c) 100 ppm sulfur dioxide and 400 ppm hydroquinone; and
(d) the plasticizer is 25 weight percent of DOP.

38. The cyanoacrylate adhesive composition of claim 35, wherein the composition comprises:
(a) weight percent of n-butyl cyanoacrylate ester;
(b) 0.1 to 1.0 weight percent of phenol;
(c) 150 sulfur dioxide and 500 ppm hydroquinone; and
(d) the plasticizer is 20 weight percent of tributyl acetyl citrate.

39. The cyanoacrylate adhesive composition of claim 34, wherein the composition comprises:
(a) a blend of decyl cyanoacrylate ester and n-butyl cyanoacrylate ester;
(b) 0.1 to 1.0 weight percent of phenol;
(c) a blend of sulfur dioxide and hydroquinone; and
(d) no plasticizer.

40. The cyanoacrylate adhesive composition of claim 39, wherein the composition comprises:
(a) 40 weight percent of decyl cyanoacrylate ester and 60 weight percent of n-butyl cyanoacrylate ester;
(b) 0.1 to 1.0 weight percent of phenol;
(c) a blend of about 150 ppm sulfur dioxide and 1000 ppm BHA; and
(d) no plasticizer.

41. The cyanoacrylate adhesive composition of claim 34, wherein the composition comprises:
(a) a blend of decyl cyanoacrylate ester and ethoxyethyl cyanoacrylate ester;
(b) to 1.0 weight percent of phenol;
(c) a blend of sulfur dioxide and hydroquinone; and
(d) no plasticizer.

42. The cyanoacrylate adhesive composition of claim 41, wherein the composition comprises:
(a) 50 weight percent of decyl cyanoacrylate ester and 50 weight percent of ethoxyethyl cyanoacrylate ester;
(b) 0.1 to 1.0 weight percent of phenol;
(c) about 100 ppm sulfur dioxide and 100 ppm hydroquinone; and
(d) no plasticizer.

43. A cyanoacrylate adhesive composition comprising:
(a) a polymerizable cyanoacrylate ester;
(b) from about 0.5 to about 1.0 weight percent phenol based on the total weight of the composition;
(c) from about 500 to about 3,500 ppm of polymerization inhibitor, and
(d) an optional biocompatible plasticizer.
